(19) United States
(12) Patent Application Publication
(10) Pub. No.: US 2025/0391025 A1
(43) Pub. Date: Dec. 25, 2025

(54) NORMATIVE REFERENCE MODEL AND USES

(71) Applicants: National University of Singapore, Singapore (SG); The University of Melbourne, Victoria (AU)

(72) Inventors: Boon Thye Thomas YEO, Singapore (SG); Sina MANSOUR LAKOURAJ, Singapore (SG); Juan ZHOU, Singapore (SG); Li-Hsian Christopher CHEN, Singapore (SG); Andrew ZALESKY, Victoria (AU); Maria Angelique DI BIASE, Victoria (AU)

(73) Assignees: National University of Singapore, Singapore (SG); The University of Melbourne, Victoria (AU)

(21) Appl. No.: 19/247,482

(22) Filed: Jun. 24, 2025

(30) Foreign Application Priority Data
Jun. 24, 2024 (SG) 10202401839Y

(51) Int. Cl. G06T 7/00; G06T 7/62; G06T 7/68

(52) U.S. Cl. CPC G06T 7/0014; G06T 7/62; G06T 7/68; G06T 2207/20076; G06T 2207/30016; G06T 2207/30061; G06T 2207/30104

(57) ABSTRACT

A system for generating a normative reference model is described. The system constructs spatial basis sets from medical scans, each basis set characterizing spatial property across the body part. A normative basis model is then generated for each spatial basis set, and a normative cross-basis model is generated from statistical relationships between the spatial basis sets. Thereafter, a normative reference model is generated from the normative basis models and the normative cross-basis model.

### TECHNICAL FIELD

The present invention relates, in general terms, to a system, and method implemented by that system, for generating a normative reference model of a body part.

### BACKGROUND

Recent brain charting studies have derived low-resolution normative charts for grey matter volume from large-scale biobanks of MRI imaging. Early works have shown promise in detecting abnormal changes in development and aging that could be an early marker of pathology. However, these approaches have limitations in capturing detailed high-resolution anatomical or functional features due to computational cost.

In an attempt to swiftly determine organ health, normative models have been proposed. To reduce computational cost, normative models are limited to coarse measurements, such as total brain volume, at resolutions on the scale of tens of centimetres. Even recent advancements claiming improved spatial resolution have typically been limited to the scale of centimetres. However, subtle individual deviations from healthy norms can manifest as localized abnormalities (at mm-scale), which can be overlooked by previous cm-scale approaches.

Existing normative modelling approaches rely on predefined sets of coarse imaging characteristics, such as total organ volume, to derive normative ranges of healthy variation. Even in the highest resolution study to-date, normative charts are computed at the centimetre-scale. Any region of interest of the organ that fall between the centimetre-scale data points are unable to be computed. These normative charts cannot be adapted for a new region of interest or a spatial query that does not align with the data points in the normative model, except by re-compute the chart with reference to the new region of interest. This re-computation is not only very slow, but also requires access to the original databank (which might be proprietary) used to compute the original normative model. Moreover, where there is a scarcity of data, significant data for some age groups but not others, or new imaging modalities become available, these models are inherently inaccurate or outdated.

It would be desirable to overcome at least one of the above-described problems by providing a means for reliably producing a normative model that is computationally efficient and can be used to infer organ health in regions that were not previously computed.

### SUMMARY

In view of the above problems, disclosed is a novel method for generating highly flexible, high-resolution normative charts applicable to a wide range of medical imaging data. The method leverages graph signal processing to establish high-resolution normative trajectories for healthy organ changes throughout the human lifespan. These normative trajectories are applicable to organs such as the brain, heart, and kidney.

Disclosed is a normative reference model for a body part, the normative reference model combining normative basis models and a normative cross-basis model, the normative basis models being constructed from respective spatial basis sets derived from medical scans of the body part, and onto which a spatial distribution of one or more biological parameters of the body part for healthy individuals are projected, and a normative cross-basis model that models statistical relationships between the spatial basis sets.

Disclosed is a method for generating a normative reference model, comprising: receiving medical scans of a particular body part for each of a plurality of individuals; constructing spatial basis sets from the medical scans, each basis set characterizing a spatial property across the body part; generating a normative basis model for each spatial basis set by projecting a spatial distribution of one or more biological parameters of healthy ones of the individuals onto the spatial basis sets; generating a normative cross-basis model from statistical relationships between the spatial basis sets; and generating a normative reference model from the normative basis models and the normative cross-basis model.

Disclosed is a method for identifying deviations in a body part of a patient, from a norm for that body part, comprising: receiving a medical scan of the body part; processing the medical scan to extract a spatial distribution of one or more biological parameters; producing a map showing deviations of a spatial distribution of the one or more biological parameters across the body part, from a spatial distribution of the one or more biological parameters across the body part for healthy individuals, by comparing the spatial distribution to the normative reference model, above.

Disclosed is a system for generating a normative reference model, comprising: memory; and at least one processor, the memory storing instructions that, when executed by the at least one processor, cause the system to: receiving medical scans of the human body part of a plurality of individuals; constructing spatial basis sets from the medical scans, each basis set characterizing spatial property across the body part; generating a normative basis model for each spatial basis set by projecting a spatial distribution of one or more biological parameters of healthy ones of the individuals onto the spatial basis sets; generating a normative cross-basis model from statistical relationships between the spatial basis sets; and generating a normative reference model from the normative basis models and the normative cross-basis model.

The terms “particular body part”, “same body part” and similar refer to the organ, or region thereof, under consideration being the same for all individuals in respect of which analysis is being undertaken. For example, a scan of the lungs of one individual will be analysed against a normative chart developed using scans of lungs of other individuals.

Advantageously, embodiments provide high-resolution normative charts. Moreover, the resolution of the normative charts increases as more data becomes available for an existing imaging modality, or a new imaging modality is developed. In some embodiments, the disclosed methods improve normative chart resolution by at least an order of magnitude over pre-existing methods. This enables identification of subtle localized deviations from healthy norms with unprecedented precision.

Advantageously, embodiments of the method are computationally efficient as they leverage low dimensional spatial basis sets (e.g., eigenmodes, Fourier bases, etc.) for dimensionality reduction. Owing to this low dimensionality, normative trajectories of this basis set can be efficiently estimated within a reasonable time.

Advantageously, embodiments enable real-time exploration of an individual's organ deviation-from-the-norm map—that is, that locations or regions in an organ, where a biological parameter of the individual deviates from that of a healthy person. That deviation may be any difference, or may need to be above a predetermined threshold, such as 5% difference.

As used herein, a “biological parameter” can be any appropriate parameter detectable using a particular imaging modality, such as cortical thickness, volumetric blood flow, symmetry or asymmetry between parts of the organ such as volumetric difference between left and right hippocampi, lung volume asymmetry between left and right lungs.

Advantageously, embodiments provide the ability to compute normative charts of a “contrast” between arbitrary regions of an organ, including previously unconsidered regions, on the fly within an individual when compared with healthy individuals. Current state-of-the-art approaches compute normative charts on a pre-defined set of regions of interest, e.g., a normative chart for right hippocampus volume and a normative chart of left hippocampus volume. In general, the chart for the left and the chart for the right will be dissociated. If a user is interested in the biological parameter of volumetric difference (i.e., asymmetry) between left and right hippocampi, prior art methods require the normative chart of this difference (or asymmetry) to be computed from scratch. Using methods disclosed herein, the normative chart of hippocampal volumetric asymmetry can be computed on the fly in real-time after taking into account the statistical dependencies among the spatial bases.

### BRIEF DESCRIPTION OF THE DRAWINGS

Embodiments of the present invention will now be described, by way of non-limiting example, with reference to the drawings in which:

FIG. 1 is a workflow diagram of highly-flexible high-resolution normative charting for medical imaging data in accordance with present teachings.

FIG. 2 illustrates a workflow of an embodiment of the present invention for highly-flexible high-resolution normative brain charts.

FIG. 3 illustrates, in image (A), an example basis set (connectome eigenmodes) used for low dimensional representation of high-resolution information over the cortical surface and, in image (B), the covariance matrix used to model statistical dependencies among bases.

FIG. 4 shows: image (A) Normative trajectories of average cortical thickness; image (B) Coarse-scale deviation-from-the-norm cortical thickness estimates for the same individual to whom the highlighted mark in image (A) applies; image (C) shows that normative ranges can be estimated at high resolution for any individual given the age and sex of the individual; image (D) is a high-resolution (mm-scale) deviation-from-the-norm cortical thickness estimate for the same individual as the marker shown in image (A), developed using the present methods; image (E) shows the total compute time for state-of-the-art approaches versus the present approach, for estimating high-resolution (mm-scale) deviation-from-the-norm cortical thickness map for the individual in panel D; and image (F) shows the same information is image (E), but on the log-scale.

FIG. 5 illustrates use of the present methods to generate a normative chart for any new region of interest (or spatial query) in real time, in which: image (A) provides a high-resolution deviation-from-the-norm thickness asymmetry map, generated on the fly for the individual for whom the estimate in FIG. 4 image (C) was generated; image (B) is a comparison of the computation time for state-of-the-art approaches for generating FIG. 5 image (A), when compared with the computation time for the present method; and image (C) presents the same information as image (B), but on the log-scale.

FIG. 6, comprising images (A) and (B), illustrates the approach for two individually-defined functional networks.

FIG. 7 illustrates a computer system for executing the methods of FIGS. 1 and 2.

### DETAILED DESCRIPTION

Brain charting processes typically derive low-resolution normative charts for grey matter volume from large-scale biobanks of MRI images. Brain charting has the potential for use in detecting abnormal changes in development and aging that could be an early marker of pathology. Known approaches have limitations in capturing detailed high-resolution anatomical or functional features due to computational cost and availability of data—e.g., where information about a change in a particular region is sought, but the stored MRI images lack data directly applicable to that particular region.

The proposed method is capable of efficiently overcoming existing limitations, enabling the estimation of normative charts with high spatial precision. Here, “high spatial precision” refers to the resolution of the imaging modality. For example, current in-vivo MRI technology typically image organs at millimetre scale, and thus high-resolution refers to the millimetre scale. If a future imaging modality can image a human organ at the micrometre scale, the present approach can be used to estimate normative models at the micrometre scale (instead of just millimetre scale).

Furthermore, once the normative charts are estimated, the present approach allows highly flexible high-resolution quantitative exploration of an individual's organ's imaging markers (e.g., cortical thickness) with very little additional computational cost. This method can potentially enable personalized medical prognosis, diagnosis, and intervention by early-stage detection of subtle abnormalities indicative of pathological deviations from healthy norms.

The high-resolution normative charts open new avenues for precise early detection, diagnosis, and intervention at the individual-level.

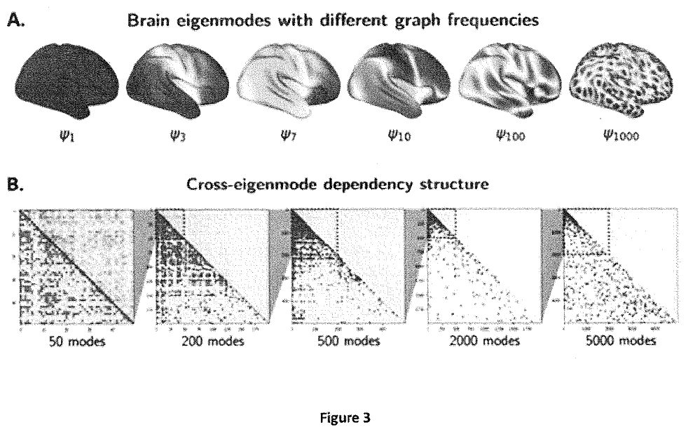

The present approach works by generating normative basis models. A “normative basis model” is a model describing the appearance or other features (e.g., volume) of one or more organs (e.g., the brain or lungs) under a particular imaging modality. A normative basis model (also referred to as a normative reference model) is constructed from a spatial basis set derived from a medical scan of an organ or other body part, onto which biological parameters for healthy individuals are projected. The spatial basis set is a set of descriptors—e.g., a descriptor for a particular body part (such as the left cortex or right cortex), parameters of the body part at different locations (such as cortical thickness at different locations), relationships between the body part at different locations (e.g., relative cortical thicknesses at different locations), descriptors for different brain areas and the like. This basis set reduces the complexity of high-dimensional imaging data to a lower dimensional latent space. It is envisaged that a high resolution input data set (i.e., higher solution input images) will require a larger spatial basis set. Basically, the Laplacian eigenmodes are naturally sorted so the lower eigenmodes capture lower frequency (lower resolution), while the higher eigenmodes capture higher frequency (higher resolution)—this is observable in FIG. 3A. Therefore, if the imaging data is higher resolution, the same approach can be used but with more eigenmodes than for lower resolution. So for example, instead of 1000 shown in FIG. 3A, we might need to use more than 1000 eigenmodes.

In general, a normative basis model will also be a function affected by different properties, such as the particular gender, and the particular age or age range, and/or show changes in the one or more organs over time (i.e., as a patient ages). A normative model may include a range for one or more metrics and containing a predetermined percentage of all data used in generation of the normative basis model—e.g., for hippocampal volume, all healthy hippocampal volumes for middle aged (40-49 years old) males may be between 3.45 cm³ and 3.64 cm³ in which case the normative basis model may show the median (e.g., 3.54 cm³) the average (e.g., 3.53 cm³) and/or a range 3.45 cm³ and 3.64 cm³. The range may contain less than 100% of all healthy training data, such as 80% or 90%.

Normative basis models are generated using images, of the one or more organs, from the particular imaging modality for healthy individuals.

A “normative cross-basis model” describes a statistical relationship between normative models. This can similarly be referred to as modelling statistical relationships between spatial basis sets forming the basis for respective normative basis models. A normative cross-basis model allows one normative basis model to provide information about another normative basis model. For example, there may be one normative basis model describing the cortical thickness of the left dorsolateral prefrontal cortex (DLPFC) for males of various ages, a second normative basis model describing cortical thickness of the right DLPFC for males of various ages, and a normative cross-basis model describing the statistical relationship between normative basis models of cortical thickness of the left and right DLPFC for males (or any arbitrary region or regions of the brain or other organ)—this can include spectral information, raw information derived from the images, and others. A “spatial basis set” thus refers to a set (group) of spatial bases, each spatial basis explains a particular characteristic in the body part. If a new image is received for a male aged in the range of 35-39 years old, the normative basis model for left and right DLPFC may be used along with their respective normative cross-basis model to determine the cortical thickness ranges expected for average DLPFC thickness (considering left and right DLPFC), and the normative cross-basis model is important to give an accurate range estimate in this example.

By generating a normative cross-basis model, the normative models can then be combined arbitrarily (with minimum computational cost) to generate or estimate a normative basis model (e.g., a chart) for any new region of interest, or to respond to a spatial query, for a desired imaging modality. The combination of normative ranges over bases (i.e., normative basis models) is non-trivial and involves accounting for the statistical dependencies among the bases (e.g., dependencies between eigenmodes). The present approach affords rapid estimation of high-resolution normative organ charts across the lifespan of an individual. Consequently, the present approaches affords computation of a high-resolution deviation-from-the-norm map of a new individual in a very short period of time—i.e., a map generated by comparing each location of an organ in the new individual to that same location in the organ of the high-resolution normative organ chart (i.e., normative basis model), the map highlighting locations or regions at which the deviation of the individual from the normative basis model is above a predetermined threshold (including greater than 0% deviation, greater than 5% deviation or some other threshold). The computational efficiency also enables real-time exploration of an individual's deviation-from-the-norm map, and avoids the need for access to the original databank used to calculate the normative basis models.

The present methods make it possible for end users (e.g., clinicians, patients, etc.) to explore the deviation-from-the-norm organ map of an individual by specifying new regions of interest (or spatial query) on the fly. Moreover, new regions of interest can potentially be defined based on an imaging modality that is different from the normative basis model desired for the spatial query or region of interest. This is especially useful in organs, such as the brain, where different regions of interest are of interest to different users. For example, some users might be interested in anatomically defined regions of interest, requiring a first imaging modality, while others might be interested in functionally defined regions of interest, requiring a second imaging modality. The first imaging modality and the normative cross-basis model defining the statistical relationship between images of the brain in the first modality and in the second modality, can be used to estimate a normative basis model for the region of interest in the second imaging modality.

Any spatial map can be expressed as a linear combination of the spatial basis sets, according to the present methods. This enables for example, the normative chart of hippocampal volumetric asymmetry to be computed on the fly in real-time after taking into account the statistical dependencies among the spatial bases. Once again, the contrast can be defined based on another imaging modality. In view of the above, a normative reference model for a body part can be arbitrarily established by combining the normative basis models and a normative cross-basis model.

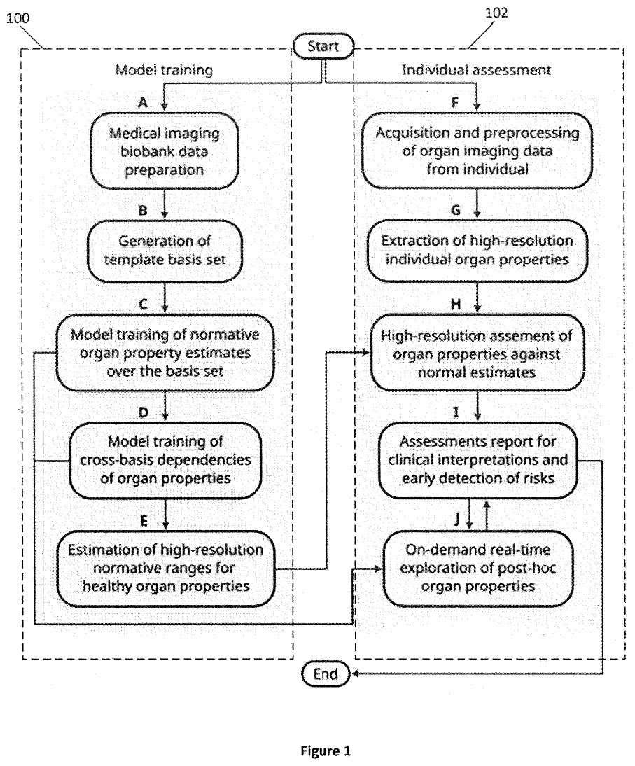

A method, represented as a schematic workflow, for generating a normative reference model and using that model to estimate a normative reference model for an individual, in real-time, is shown in FIG. 1. The method comprises a model training phase 100 during which the normative basis models and normative cross-basis model are generated, and an individual assessment phase 102 during which the models generated in phase 100 are used to estimate a normative basis model for comparison with an individual's newly acquired scan images.

In step (A) medical scans of a particular body part are obtained, for each of a plurality of individuals. To form a normative basis model from which deviations can be detected, the individuals must be healthy at least insofar as the organ shown in the medical scans is concerned. The medical scans may be obtained by MRI, fMRI and various other imaging modalities. In addition, the medical scans include patient information such as age and gender.

The medical scans may be obtained from an imaging biobank of a certain organ, formed by collecting and combining several organ imaging datasets. As set out above, this biobank will include healthy organ images of a diverse age range to adequately cover the human lifespan. All imaging data will be pre-processed and aligned to a standard template space to facilitate cross-participant comparison. For example, all imaging data will be normalised to a common reference frame, such as through RGB or pixel intensity normalisation, size normalisation (e.g., where a pixel represents a predetermined area in the actual organ), intensity thresholding, noise removal and other processes.

In step (B) spatial basis sets are constructed from the medical scans obtained in Step (A). Each spatial basis characterizes a spatial property across the body part—e.g., defines the relationship of one region of an organ to another region. The spatial basis sets may be developed by generating a spatial representation of the organ. The spatial representation may be a graph representation, grid representation or another type of representation defining the spatial relationship between regions of the organ. In the case of the brain, this graph representation may correspond to the connectivity matrix of the brain. In another organ (e.g., liver), the representation may correspond to a tetrahedral mesh representing the anatomy of the organ. A graph Laplacian matrix, or graph Laplacian, can then be generated to describe the relationship between nodes in the representation. A comprehensive spatial basis set can be generated by decomposing the graph Laplacian matrix to singular values. The decomposition will identify the eigenvectors and eigenvalues, and eigenmodes can be derived from this decomposition in a known manner. The eigenmodes act as a low-dimensionality spatial basis set. Any spatial pattern or region of interest can be efficiently approximated using this basis set, to represent biological (e.g., anatomical, physiological, genetic) behaviour of the body part being analysed.

An alternative non-graph basis set (e.g., principal components or Fourier basis or wavelet basis) can also be used. For instance, performing principal component analysis (PCA) of the high-resolution imaging data acquired from a body part over the training sample can similarly produce eigenvalues and eigenvectors (eigenmodes) that form a different spatial basis set. These PCA modes are not based on graph Laplacian decomposition, but can be a valid alternative when an appropriate graph representation is not available for the imaging data from a body part.

Under Step (C), a normative basis model is then constructed for each spatial basis set. This is achieved by projecting one or more biological parameters of healthy ones of the individuals, for whom the medical scans were obtained under Step (A), onto the spatial basis sets. For example, an imaging marker for each healthy individual within the imaging biobank can be projected onto the spatial basis set. In some embodiments, this involves specifying a dimension of an organ (which includes part of an organ), such as the thickness of the left or right hippocampus, at each node in the spatial basis set, or specifying blood flow at each node in the spatial basis set. The biological parameters may include the patient information, such as age and gender, obtained in Step (A). A standard normative modelling method can then be used to derive normative ranges for each spatial basis, with respect to the biological parameters. Notably, any normative modelling method can be used in this step (e.g., hierarchical Bayesian regression, Gaussian process regression or Bayesian linear regression or neural process and network models). Notably, the imaging marker is organ dependent.

After Step (C), a normative basis model has been estimated for every basis—e.g., imaging modality, age, gender, etc. Given a newly defined brain region of interest (or spatial query), it is desirable to compute a normative model for this region of interest (or spatial query). However, this is not trivial because the organ properties modelled (in Step C) are not independent between any given pair of bases. Given the complex cross-basis statistical dependencies, under Step (D), a normative cross-basis model is generated from statistical relationships between the spatial basis sets. This is done by deriving a sparse representation of cross-basis dependencies—i.e., dependencies between the normative basis models, depending on the biological parameters. The sparse representation improves model accuracy on any arbitrary region of interest (or spatial query) involving multiple spatial bases-sparse matrix representation is discussed in relation to FIG. 3B. The sparse representation can be used for accurate and rapid computation of a normative model for any arbitrary region of interest (see Steps (E) and (J) below). Importantly, the normative model for the arbitrarily selected region can be generated without requiring access to the original databank.

Under Step (E), the models from Steps (C) and (D), that have been fitted to the data of healthy individuals, are combined to generate a (high-resolution) normative reference model (or normative reference chart) of the imaging marker for the organ being analysed. This chart can specify single values indicating health, or can include a normative range for the imaging marker of the organ at every spatial location of the spatial basis set, as a function of the demographics (e.g., age, sex, and race) of the imaged participants.

Under phase 100, flexible normative charts are created for healthy organ imaging markers over the human lifespan. Up to this point, all computations are only performed once and can then be used to assess deviation-from-the-norm of a new individual's imaging markers. These assessments form phase 102:

Under step (F), a medical scan of a body part in a new individual is received. This medical scan will generally be organ imaging data (e.g., medical scans, images, etc) collected from the new individual, to be assessed for deviation-from-the-norm with respect to the normative charts computed in phase 100. Any imaging modality can be used. In addition, the medical scan will include biological parameters such as age, gender and the like.

Under Step (G), the imaging data obtained at Step (F) is processed to extract a spatial distribution of the one or more biological parameters—i.e., a map. In some embodiments, this involves extracting an individual organ imaging marker (consistent with Step (C)). The imaging marker is then mapped map onto the template space—i.e., the relevant spatial basis set established at Step (B).

Under Step (H), using the individual's imaging marker map generated at Step (G) and the high-resolution normative estimates from Step (E), a map can be produced, showing deviations of a spatial distribution of the one or more biological parameters across the body part, from a spatial distribution of the one or more biological parameters across the body part for healthy individuals. This is done by simply comparing the spatial distribution for the individual, to the normative reference model, at each node in the map.

The result is a deviation-from-the-norm imaging marker map for the individual. In some embodiments, this map shows, for each spatial location of the organ (at the mm-scale or higher resolution depending on the imaging technology), at what normative percentile the individual ranks in terms of the imaging marker. Depending on the imaging marker, a very low and/or very high percentile could indicate abnormality. Thus, given phase 100, Steps (F) to (H) provide a method for identifying deviations in a body part of a patient, from healthy norms for that body part.

Under Step (I), health of the body part is determined from the map of deviations generated at Step (H). The assessments (i.e., deviations from the norm, determined under Step (H)), or assessed organ health, are reported back to the clinician (or patient or other end-user) for interpretation. These assessments could also include risk scores for various organ diseases whose symptoms include deviations from healthy organ imaging marker charts. For example, a deviation above a first predetermined threshold may indicate minor risk of a particular condition. Above a second predetermined threshold, the individual may be at critical risk of a particular condition.

Under Step (J), the end user (clinician, patient, etc.) can explore the individual's deviation-from-the-norm model by specifying a new region of interest (or spatial query). Normative models (from Step (C)) can then be used to generate a normative chart for this new region of interest after accounting for statistical dependencies between basis pairs (from Step D) in real time. This step can be repeated as many times as the end user wishes to. This new region of interest (or spatial query) can be based on another imaging modality.

FIG. 1 describes an approach for computing normative charts for a spatial basis set. The normative chart for any new region of interest (or spatial query) can be computed in real time by combining normative models across the bases, while accounting for the complex statistical dependencies across bases. The approach of FIG. 1 allows the computation of a high-resolution deviation-from-the-norm organ map of an individual. Here, “high-resolution” refers to the resolution of the imaging modality. For example, current in-vivo MRI technology typically image organs at millimetre scale. If a future imaging modality can image a human organ at a micrometre scale, our approach can be easily used to estimate normative models at the micrometre scale (instead of just millimetre scale).

The present technology will enable powerful screening tools across the lifespan of patients. Oftentimes, each body organ is treated separately in the clinic, as can be seen by separate medical specialties focusing on different organs. However, all of a patient's organs must work together to keep them alive and functioning normally. Using the present technology, high-resolution normative charts can be generated for multiple organs. These charts can then be used to estimate a high-resolution deviation-from-the-norm map for every organ of an individual, thus providing a universal screening tool to detect whether any certain organ system is abnormal for the individual.

To illustrate the applications of the present technology in the context of brain health, the present approach is applicable for:
(A) Early detection of abnormal brain development in children, which complement current highly coarse charts of head size measurements, weight, and height used in kids.
(B) Early detection of accelerated aging (e.g., for early dementia risk evaluation) and risk susceptibility to several types of dementia based on a personalized pattern of brain abnormalities.
(C) Early detection of general abnormalities, e.g., from traumatic brain injury or stroke. The brain is a complex network with widespread connections between brain regions. Stroke in a localized brain region can lead to abnormalities beyond the stroke location, damaging regions far away from the stroke lesion. These downstream regional damages are much more subtle than the stroke lesion itself and could be easily missed by the neurologist. Our high-resolution normative charts can potentially detect these subtle damages.

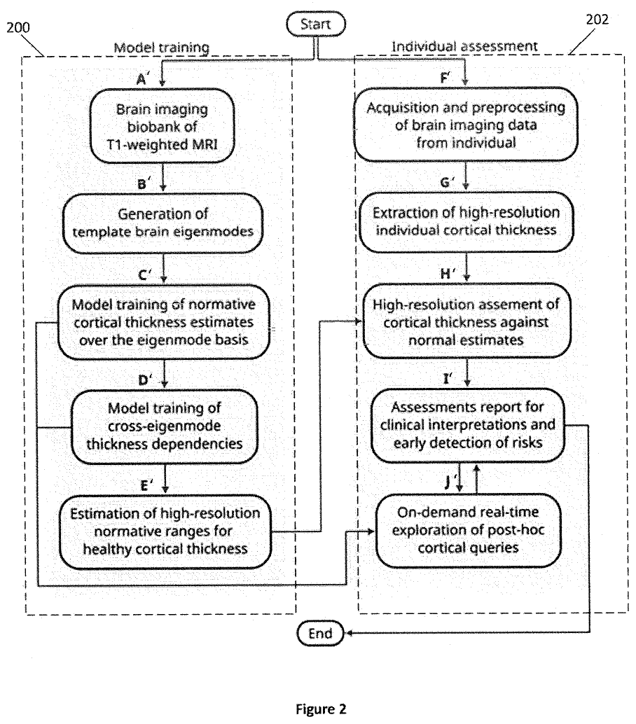

FIG. 2 illustrates the instantiation of the approach of FIG. 1 for individualized assessment of abnormalities based on high-resolution normative charts of cortical thickness.

Step (A′)—a brain imaging biobank of T1-weighted structural MRI is collected by combining several brain imaging datasets. This biobank includes healthy brain images of a diverse age range to adequately cover the human lifespan. All imaging data are pre-processed and aligned to a standard template space to facilitate cross-participant comparison.

Step (B′)—a high-resolution connectivity matrix is generated from consensus connectivity maps encompassing major anatomical pathways of the brain. This connectivity matrix is utilized to construct a comprehensive spatial basis set by generation of brain eigenmodes via spectral decomposition of the graph Laplacian matrix. These eigenmodes (FIG. 3, image (A)) act as a low-dimensionality spatial basis set. Any spatial pattern or region of interest can be efficiently approximated using the basis set.

Step (C′)—cortical thickness of all healthy individuals within the imaging biobank are projected onto the eigenmode bases. A normative modelling method based on hierarchical Bayesian regression is used to derive normative ranges for each eigenmode basis while also harmonizing cortical property biases introduced by protocol differences across sites, by modeling and removing the cross-site differences as a hierarchical random confounding effect.

Step (D′)—after Step (C′), a normative model is estimated for every eigenmode basis. Given a newly defined brain region of interest (or spatial query), a normative model will be computed for this region of interest (or spatial query). However, this is not trivial because the cortical properties modelled (in Step (C′)) are not independent between any given basis pair. Given the complex cross-basis statistical dependencies, a sparse representation of cross-basis dependencies is derived to improve model accuracy on any arbitrary region of interest (or spatial query) involving multiple spatial bases (FIG. 3, image (B)). This allows accurate and rapid computation of a normative model for any arbitrary region of interest (see Step (J′) below) without access to the original databank (which could be proprietary).

Step (E′)—the fitted models from Step (C′) and (D′) are then combined to generate a high-resolution normative reference chart of cortical thickness. This chart includes a normative range for the thickness of every spatial coordinate (i.e., millimetre resolution) on the cortical surface as a function of the demographics (e.g., age, sex, and race) of the imaged participants.

The steps in phase 200 form the first, training stage that creates flexible normative charts of healthy cortical thickness over the human lifespan. Per method 100, all computations required for this stage are only performed once and can then be used to assess deviation-from-the-norm of a new individual's cortical thickness. These assessments form phase 202.

Step (F′)—structural brain MRI is collected from a new individual to be assessed for deviation-from-the-norm with respect to the normative charts computed from stage 200.

Step (G′)—the MRI data will be processed to extract individual cortical thickness and map onto the template surface.

Step (H′)—using the individual's cortical thickness map (Step (G′)) and the high-resolution normative estimates (from Step (E′)), a high-resolution deviation-from-the-norm cortical thickness map can be produced for the individual. This map will show, for each spatial coordinate on the cortex (at the mm-scale), at what percentile the individual ranks in terms of cortical thickness.

Step (I′)—these assessments are reported back to the clinician (or patient or other end-user) for interpretation. These assessments could also include risk scores for various brain diseases or mental health issues whose symptoms include deviations from healthy cortical thickness charts (e.g., dementia). Risk scores can be based on an amount of deviation of a patient's biological parameters from the norm.

Step (J′)—the end user (clinician, patient, etc.) can explore the individual's deviation-from-the-norm model by specifying a new region of interest (or spatial query). Normative models (from Step (C′)) can then be used to generate a normative chart for this new region of interest after accounting for statistical dependencies between basis pairs (from Step (D′)) in real time. This step can be repeated as many times as the end user wishes to. This new region of interest (or spatial query) can be based on another imaging modality, e.g., individualized resting-state networks from resting-state functional MRI.

### Experimental Setup

A comparative analysis was performed, to highlight the capabilities of the present technology on an exemplary application for estimating normative charts of cortical thickness. To this end, brain imaging data was sourced from the Human Connectome Project (HCP). Three distinct biobanks were combined, to cover a wide range of human lifespan comprising developing (HCP-D), young adult (HCP-YA), and aging (HCP-A) cohorts. For all cohorts, pre-processed T1-weighted structural images were registered to a surface template and cortical thickness was estimated.

The goal of this analysis is to illustrate the advantages of the present methodology with respect to the state-of-the-art existing methods. Specifically, the present approach will be compared with two existing approaches: (1) Bethlehem2022 normative ranges for mean cortical thickness, and (2) Rutherford2022 normative ranges for regional cortical thickness.
1. Similar to Bethlehem2022, the normative chart for cortical thickness averaged across the cerebral cortex was estimated using hierarchical Bayesian regression.
2. Similar to Rutherford2022, normative charts of 148 different brain regions from the Destrieux brain atlas were estimated using hierarchical Bayesian regression. Of note, Rutherford2022 represents the highest resolution achieved by previous works and is included in the present evaluation to underscore the significant leap in spatial resolution of the methods described herein.

For present purposes, the model uses the eigenmode decomposition of the high-resolution consensus connectome mapped for the Human Connectome Project data with the 1000 modes. Hierarchical Bayesian regression is used to estimate the normative chart for each eigenmode.

The same method was used for all three approaches for normative model fitting for fair comparison. By using the same normative modelling fitting procedure, any improvements in computational time observed in the present approach are solely attributed to the spatial basis set combination technique described herein. This ensures that similar relative improvements can be achieved even if future normative modelling techniques enhance efficiency, as the performance enhancements primarily stem from the basis set combination methodology.

### Results

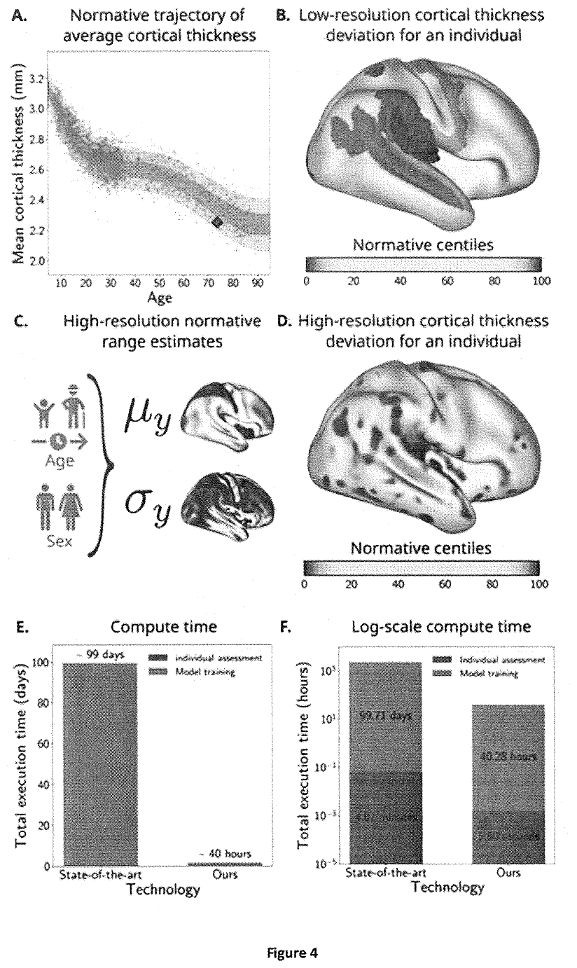

With reference to FIG. 4, image (A) shows the normative average cortical thickness trajectory across the lifespan. The three shades of (from darkest to lightest) represent the [25-75], [5-95], and [1-99] centile ranges, respectively. The bold marker indicates the mean thickness of an example individual's brain or cerebral cortex. While the Bethlehem2022 approach can indicate that this individual has globally thinner cortical grey matter compared to healthy norms (falling in the 13^{th} centile), it does not provide information regarding the locations in which this thinning is occurring. FIG. 4, image (B) demonstrates that the Rutherford2022 approach can provide coarse-scale deviation-from-the-norm estimates for the same individual as for whom the brain in Image (A) is shown, revealing a low-resolution map of regional thickness abnormalities specific to the individual.

As depicted in FIG. 4, image (C), the present approach estimates normative ranges for the same individual at a significantly higher spatial resolution, achieving millimetre accuracy. FIG. 4, image (D) shows the high-resolution deviation-from-the-norm maps for the same individual, generated using the method of FIG. 1, highlighting the increase in spatial resolution achieved using the present methods over the prior art. These results highlight the presence of brain regions exhibiting large, but spatially local, deviation-from-the-norm in cortical thickness, which are either overlooked in Bethlehem2022 or incorrectly attributed to larger brain regions in the coarse-scale deviation-from-the-norm map of Rutherford2022 (FIG. 4, image (B)).

To compute the high-resolution deviation-from-the-norm map from FIG. 4, image (D), the traditional approach (Bethlehem2022; Rutherford2022) would have required around 99 days of computation time on a single CPU (FIG. 4, images (E) and (F)). The present approach requires only 40 hours with most of the time spent on training the original normative models (40.28 hours) and around 5 seconds to generate the high-resolution deviation-from-the-norm map for a new individual. Thus, once the original normative models are generated, the computer time for new queries is a matter of seconds, as compared with months.

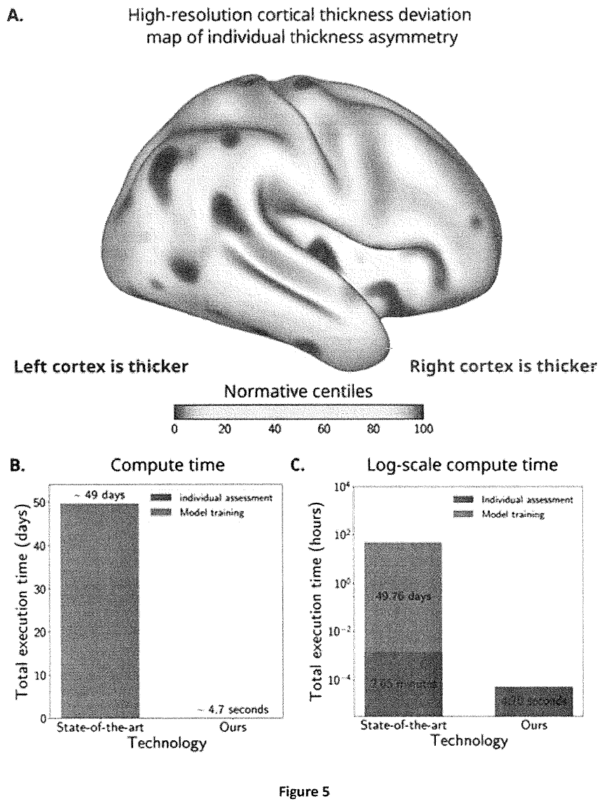

With reference to FIG. 5: the present approach can be used to generate a normative chart for any new region of interest (or spatial query) in real time, thus providing a quantitative deviation-from-the-norm map on the fly. FIG. 5, image (A) shows a high-resolution deviation-from-the-norm thickness asymmetry map of an individual (from FIG. 4) generated by the method of FIG. 1, on the fly. The map shows that for this individual, the cortical thickness asymmetry is abnormally high or low, relative to the population norm at the individual's age and sex, at specific brain locations.

As shown in FIGS. 5, images (B) and (C), this asymmetry map would have required 49 days of computational time for the traditional approach (Bethlehem2022; Rutherford2022), while only requiring 4.7 seconds with the present method. Note that 4.7 seconds might not be considered real time, but it is expected that actual real time can be achieved with implementation in a commercial application/software.

The reason for this massive speed improvement is that traditional approaches require fitting the normative models from scratch for any new region of interest (or spatial query). This is not only slow but requires access to the original databank, which may be proprietary or no longer accessible. However, with the present approach, the normative models estimated for the spatial basis set can be reused and combined while accounting for statistical dependencies between the bases to generate a normative chart and an individualized deviation-from-the-norm map for the new region of interest (or spatial query) very rapidly.

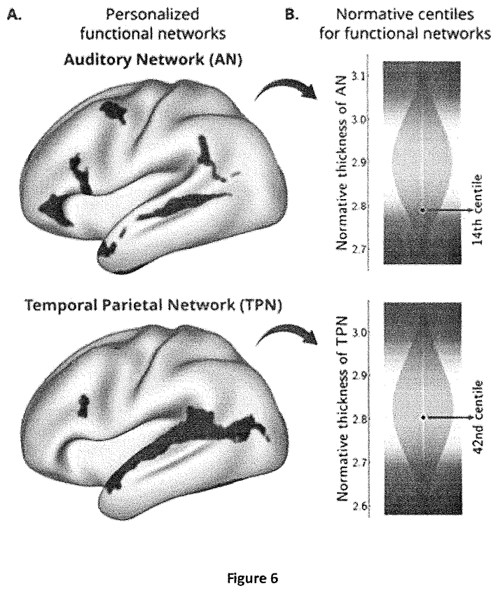

FIG. 6 illustrates advantages of the present approach for two individually-defined functional networks (FIG. 6, image (A)), namely the auditory network (AN) and the temporal parietal network (TPN)) estimated from the resting-state fMRI of a 24 year old female individual. These networks were generated to demonstrate application of the present method to personalized data. FIG. 6, image (B) shows that the present approach can evaluate normative deviations of thickness in personalized regions. This example shows that the thickness of this individual's TPN is on the 42^{nd} percentile (within healthy ranges) compared with other individuals of the same age and sex. In contrast, the thickness of this individual's AN is in the 14^{th} percentile which indicates considerable thinning of AN relative to other individuals of the same age and sex. The computational time for our approach is 145 milliseconds.

Traditional approaches are fundamentally limited due to a lack of access to the personalized brain network of this individual during training of the normative models. Therefore, in order to generate FIG. 6, image (B), the traditional approaches would require the end user to have access to the original imaging database to compute a normative model from scratch, which fundamentally limits the application of the traditional approach to this use case.

### (1) Basis Set Derivation

Ideas from graph signal processing are used to perform graph Laplacian decomposition of a connectivity matrix to form a spatial basis for graph Fourier transformation. Namely, from a high-resolution connectivity matrix A (with a diagonal degree matrix D), the random walk Laplacian L is constructed as follows:

$$L = {L}^{rw} = I - {D}^{- 1} A$$

This Laplacian matrix can be decomposed using a Singular Value Decomposition (SVD) to generate brain connectivity eigenmodes (FIG. 3, image (A)):

$$L = V {ΛV}^{- 1}$$

These eigenmodes can be used to encode a brain signal (contrast/map), denoted by X, to graph frequency domain (a Graph Fourier Transform, GFT):

$$\vec{\tilde{X}} = {V}^{T} \vec{X}$$

Importantly, the original signal can be reconstructed from its graph frequency domain encoding:

$$\vec{X} = V \vec{\tilde{X}}$$

A partial decomposition of the graph Laplacian can be used to approximate a low-pass reconstruction by the first NLP=1000 eigenmodes:

$$( {V}_{LP} ∈ {R}^{{N}_{v}} \times {R}^{{N}_{LP}} ) .$$

### (2) Normative Modelling Using the Eigenmode Basis Set

Using a normative modelling method (e.g., Hierarchical Bayesian Regression) normative ranges can be estimated for each eigenmode basis:

$${\tilde{T}}_{LP} ( i ) ∼ N ( {μ}_{i} , {σ}_{i} )$$

$${μ}_{i} = {f}_{{μ}_{i}} ( age , sex , site )$$

$${σ}_{i} = {f}_{{σ}_{i}} ( age , sex , site )$$

Here, \tilde{T}_{LP}(i) denotes the i^{th} row of TLP and is the vector that contains the thickness loading of the i^{th} eigenmode for all individuals. This loading is modelled as a normative characteristic of individuals that changes as a function of age, gender, and site.

### (3) Modelling Cross-Basis Dependencies

A sparse matrix representation of the covariance structure across basis sets is then fitted to model cross-basis dependencies in a computationally efficient and accurate approach. First, a limited set of eigenmode pairs are selected. The selection is based on a predetermined magnitude threshold over observed cross-basis correlations within the training data (e.g., ρ>0.25). An example of this sparse subset selection is presented in FIG. 3, image (B). Thereafter, for every pair within this subset, pairwise dependencies are modelled as a function of age, sex, and site with a normative modelling approach similar to that of “(2) Normative modelling using the eigenmode basis set”. This dependency structure between a pair of variables, representing thickness loadings over a pair of eigenmodes, can be presented in either covariation or correlation matrix form:

$$\begin{matrix}Cov ( {\tilde{T}}_{LP} ( i ) , {\tilde{T}}_{LP} ( j ) ) = \sqrt{Var ( {\tilde{T}}_{LP} ( i ) )} \sqrt{Var ( {\tilde{T}}_{LP} ( j ) )} Corr ( {\tilde{T}}_{LP} ( i ) , {\tilde{T}}_{LP} ( j ) ) \\ = {σ}_{i} {σ}_{j} Corr ( {\tilde{T}}_{LP} ( i ) , {\tilde{T}}_{LP} ( j ) )\end{matrix}$$

This covariance structure can also be denoted by the following linear algebraic notation:

$$cov ( {\tilde{T}}_{LP} ) = {K}_{TT} = {( diag ( {K}_{TT} ) )}^{\frac{1}{2}} corr ( {\tilde{T}}_{LP} ) {( diag ( {K}_{TT} ) )}^{\frac{1}{2}}$$

Where K_{TT} denotes the covariance matrix (dependency structure presented in FIG. 3, image (B)) for which the diagonal elements (variance of eigenmodes) are estimated in “(2) Normative modelling using the eigenmode basis set”. The off-diagonal elements of this matrix are reduced to a sparse set of covariance pairs (via thresholding) that are modelled to approximate the full covariance structure.

### (4) Combining Eigenmode Normative Charts

After estimation of separate normative charts for the eigenmode basis as well as a sparse cross-basis dependency (i.e., covariance) structure, a derivation based on multivariate normal distributions is used to estimate the normative ranges (i.e., mean and standard deviation as a function of age, sex, and site) from linear combination of eigenmode normative charts.

Mean estimates can then be reconstructed for an arbitrary region of interest (spatial query X_{Q}, also denoted by T_{X} when referring to its observed values across individual samples). X_{Q} can be applied to high-resolution individual observations T to compute the observed thickness for the spatial query T_{X}:

$$\vec{{T}_{{X}_{Q}}} = \vec{{X}_{Q}} T$$

First, the query is approximated by its low pass graph spectral embedding:

$$\vec{{\tilde{X}}_{Q ( LP )}} = {V}_{LP}^{T} \vec{{X}_{Q}}$$

Then, the estimate of query mean can be achieved by the following linear combination:

$$\begin{matrix}E [ \vec{{X}_{Q ( LP )}} \cdot \vec{T} ] = E [ \vec{T} \cdot \vec{{X}_{Q ( LP )}} ] \\ = E [ \vec{T} {V}_{LP} \vec{{\tilde{X}}_{Q ( LP )}} ] \\ = E [ \vec{T} {V}_{LP} ] \vec{{\tilde{X}}_{Q ( LP )}} \\ = E [ \vec{{\tilde{T}}_{LP}} ] \vec{{\tilde{X}}_{Q ( LP )}} \\ = \vec{M} \cdot \vec{{\tilde{X}}_{Q ( LP )}}\end{matrix}$$

In other words, the following rule for combination of multivariate normal distributions is used to derive the mean estimate of the basis combination:

$$E [ {a}_{1} {X}_{1} + {a}_{2} {X}_{2} + … + {a}_{k} {X}_{k} ] = {a}_{1} E [ {X}_{1} ] + {a}_{2} E [ {X}_{2} ] + … + {a}_{k} E [ {X}_{k} ]$$

Similarly, the standard deviation of the basis combination can be approximated from the fitted basis norms and cross-dependency structure:

$$\begin{matrix}var ( \vec{{X}_{Q ( LP )}} \cdot \vec{T} ) = var ( \vec{T} \cdot \vec{{X}_{Q ( LP )}} ) \\ = var ( \vec{T} {V}_{LP} \vec{{\tilde{X}}_{Q ( LP )}} ) \\ = var ( {\vec{T}}_{LP} \cdot \vec{{\tilde{X}}_{Q ( LP )}} ) \\ = var ( \vec{{\tilde{X}}_{Q ( LP )}} \cdot \vec{{\tilde{T}}_{LP}} ) \\ = \vec{{\tilde{X}}_{Q ( LP )}} cov \vec{{\tilde{( T}}_{LP} )} \vec{{\tilde{X}}_{Q ( LP )}} \\ = \vec{{\tilde{X}}_{Q ( LP )}} diag ( std ( \vec{{\tilde{T}}_{LP}} ) corr ( \vec{{\tilde{T}}_{LP}} ) diag ( std ( \vec{{\tilde{T}}_{LP}} ) ) \vec{{\tilde{X}}_{Q ( LP )}}\end{matrix}$$

In other words, the following rule can be used to approximate the variance of a linear combination of multivariate normal distributions:

$$Var ( {\sum }_{i = 1}^{k} {a}_{i} {X}_{i} ) = ( {\sum }_{i = 1}^{k} {a}_{i}^{2} Var ( {X}_{i} ) ) + ( {\sum }_{i = 1}^{k} {\sum }_{j = 1 ( j \neq i )}^{k} Cov ( {X}_{i} , {X}_{j} ) )$$

Notably, the matrix form derivations of combination rules for basis normative charts enables efficient computation of normative chart estimates using available tensor libraries. These formulations enable parallelization of estimation tasks (over CPU/GPU) that can lead to additional speed-up for both training and assessment stages.

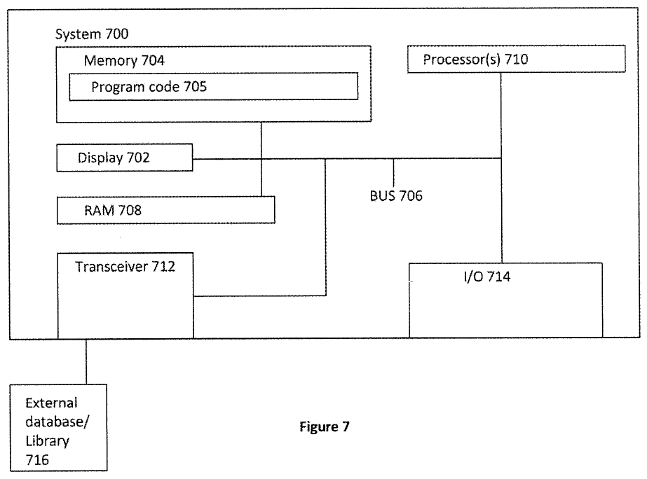

FIG. 7 schematically illustrates a system 700 for performing the methods of FIGS. 1 and 2, and as otherwise taught herein. As shown, the system comprises a mobile computer device 700 includes the following components in electronic communication via a bus 706:
(a) a display 702;
(b) non-volatile (non-transitory) memory 704 containing program code 705 for executing the method of FIG. 1 or 2;
(c) random access memory (“RAM”) 708;
(d) one or more processors (N processing components) 710;
(e) a transceiver component 712 that includes N transceivers; and
(f) user controls 714.

The system 7 interacts with a database 716 for obtaining images with which to train the normative basis models and normative cross-basis model. The database 716 may be replaced with an imaging system (e.g., MRI machine) for directly acquiring images—e.g., for new individuals.

Although the components depicted in FIG. 7 represent physical components, FIG. 7 is not intended to be a hardware diagram. Thus, many of the components depicted in FIG. 7 may be realized by common constructs or distributed among additional physical components. Moreover, it is certainly contemplated that other existing and yet-to-be developed physical components and architectures may be utilized to implement the functional components described with reference to FIG. 7.

The display 702 generally operates to provide a presentation of content to a user, and may be realized by any of a variety of displays (e.g., CRT, LCD, HDMI, micro-projector and OLED displays). For example, display 702 may display virtual buttons to a user, or may display the deviation-from-the-norm map for an individual, or other information.

In general, the non-volatile data storage 704 (also referred to as non-volatile memory) functions to store (e.g., persistently store) data and executable code. The system architecture may be implemented in memory 704, or by instructions stored in memory 704.

In some embodiments for example, the non-volatile memory 704 includes bootloader code, modem software, operating system code, file system code, and code to facilitate the implementation components, well known to those of ordinary skill in the art, which are not depicted nor described for simplicity.

In many implementations, the non-volatile memory 704 is realized by flash memory (e.g., NAND or ONENAND memory), but it is certainly contemplated that other memory types may be utilized as well. Although it may be possible to execute the code from the non-volatile memory 704, the executable code in the non-volatile memory 704 is typically loaded into RAM 708 and executed by one or more of the N processing components 710.

The N processing components 710 in connection with RAM 708 generally operate to execute the instructions stored in non-volatile memory 704. As one of ordinarily skill in the art will appreciate, the N processing components 710 may include a video processor, modem processor, DSP, graphics processing unit (GPU), and other processing components.

The transceiver component 712 includes N transceiver chains, which may be used for communicating with external devices via wireless networks or wired networks, such as database 716. Each of the N transceiver chains may represent a transceiver associated with a particular communication scheme. For example, each transceiver may correspond to protocols that are specific to local area networks, cellular networks (e.g., a CDMA network, a GPRS network, a UMTS networks), and other types of communication networks.

The system 700 of FIG. 7 may be connected to any appliance, such as one or more imaging machines to acquired images for different imaging modalities.

It should be recognized that FIG. 7 is merely exemplary and in one or more exemplary embodiments, the functions described herein may be implemented in hardware, software, firmware, or any combination thereof. If implemented in software, the functions may be stored on or transmitted over as one or more instructions or code encoded on a non-transitory computer-readable medium 704. Non-transitory computer-readable medium 704 includes both computer storage medium and communication medium including any medium that facilitates transfer of a computer program from one place to another. A storage medium may be any available medium that can be accessed by a computer.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A normative reference model for a body part comprising:
the normative reference model combining normative basis models and a normative cross-basis model, the normative basis models being constructed from respective spatial basis sets derived from medical scans of the body part, and onto which a spatial distribution of one or more biological parameters of the body part for healthy individuals are projected, and a normative cross-basis model that models statistical relationships between the spatial basis sets.

2. A method for generating a normative reference model, comprising:
receiving medical scans of a particular body part for each of a plurality of individuals;
constructing spatial basis sets from the medical scans, each basis set characterizing a spatial property across the body part;
generating a normative basis model for each spatial basis set by projecting a spatial distribution of one or more biological parameters of healthy ones of the individuals onto the spatial basis sets;
generating a normative cross-basis model from statistical relationships between the spatial basis sets; and
generating a normative reference model from the normative basis models and the normative cross-basis model.

3. The method of claim 2, wherein each medical scan is normalised to a standard template.

4. The method of claim 2, wherein the spatial basis sets represent biological (e.g., anatomical, physiological, genetic etc) behaviour of the body part.

5. The method of claim 4, wherein constructing the spatial basis sets comprises generating eigenmodes of the body part.

6. The method of claim 4, wherein constructing the spatial basis sets uses principal component, Fourier and/or wavelet analysis, of the body part.

7. The method of claim 2, wherein generating the normative models comprises performing hierarchical Bayesian regression to derive normative ranges for each basis.

8. The method of claim 2, wherein generating the normative models comprises performing one of Gaussian process regression, Bayesian linear regression, or neural process modelling, to derive normative ranges for each basis.

9. A method for identifying deviations in a body part of a patient, from a norm for that body part, comprising:
receiving a medical scan of the body part;
processing the medical scan to extract a spatial distribution of one or more biological parameters; and
producing a map showing deviations of a spatial distribution of the one or more biological parameters across the body part, from a spatial distribution of the one or more biological parameters across the body part for healthy individuals, by comparing the spatial distribution to the normative reference model of claim 1.

10. The method of claim 9, further comprising:
receiving a spatial query comprising a new region of interest not identified in the map; and
generating a normative chart for the new region of interest based on the normative basis models and normative cross-basis model.

11. A query system comprising:
memory; and
at least one processor, the memory storing instructions that, when executed by the at least one processor, cause the system to perform the method of claim 2.

12. A system for generating a normative reference model, comprising:
memory; and
at least one processor,
the memory storing instructions that, when executed by the at least one processor, cause the system to:
receiving medical scans of the human body part of a plurality of individuals;
constructing spatial basis sets from the medical scans, each basis set characterizing spatial property across the body part;
generating a normative basis model for each spatial basis set by projecting a spatial distribution of one or more biological parameters of healthy ones of the individuals onto the spatial basis sets;
generating a normative cross-basis model from statistical relationships between the spatial basis sets; and
generating a normative reference model from the normative basis models and the normative cross-basis model.

13. The system of claim 12, further configured to normalise each medical scan to a standard template.

14. The system of claim 12, wherein the spatial basis sets represent biological behaviour of the body part.

15. The system of claim 14, being configured to construct the spatial basis sets by generating eigenmodes of the body part.

16. The system of claim 14, being configured to construct the spatial basis sets by performing principal component, Fourier and/or wavelet analysis, of the body part.

17. The system of claim 12, wherein generating the normative models comprises performing hierarchical Bayesian regression to derive normative ranges for each basis.

18. The system of claim 12, wherein generating the normative models comprises performing one of Gaussian process regression, Bayesian linear regression, or neural process modelling, to derive normative ranges for each basis.

19. The system of claim 12, being configured to determine health of a body part of a patient, by:
receiving a new medical scan of the body part;
processing the new medical scan to extract a new spatial distribution of one or more biological parameters;
producing a map showing deviations of a spatial distribution of the one or more biological parameters across the body part, from a spatial distribution of the one or more biological parameters across the body part for healthy individuals, by comparing the new spatial distribution to the normative reference model of claim 1.

20. The system of claim 19, being further configured to:
receive a spatial query comprising a new region of interest not identified in the map; and
generate a normative chart for the new region of interest based on the normative basis models and normative cross-basis model.

21. The method of claim 2, wherein the normative reference model shows a variation in the one or more biological parameters with at least one of age, demographic and pathology.

22. The system of claim 12, wherein the normative reference model shows a variation in the one or more biological parameters with at least one of age, demographic and pathology.
